(19) United States
(12) Patent Application Publication
(10) Pub. No.: US 2005/0078276 A1
(43) Pub. Date: Apr. 14, 2005

(54) Head-mounting device

(76) Inventor: Chao-Chyun Lin, Pan-Chiao City (TW)

Correspondence Address: SUPREME PATENT SERVICES, POST OFFICE BOX 2339, SARATOGA, CA, 95070, US

(21) Appl. No.: 10/683,958

(22) Filed: Oct. 10, 2003

(51) Int. Cl. A61B 3/00

(52) U.S. Cl. 351/203

(57) ABSTRACT

A head-mounting device includes an elongate strip-like body having an arcuate configuration suitable to fit over a wearer's head and extending from the forehead to the back of the head whereby front and rear ends of the strip-like body are located adjacent the forehead and the back of the head. Two side-wings extend from opposite edges of the strip-like body in substantially opposite directions for engaging sides of the wearer's head. An electrically powered eye training device is attached to the front end of the strip-like body whereby the eye training device is substantially located in front of the wearer's eyes. A controller is attached to the rear end of the strip-like body and is electrically connected to the electrically powered eye training device for controlling the operation of the eye training device.

### BACKGROUND OF THE INVENTION

1. Field of the Invention

The present invention relates generally to a device for mounting to a wearer's head for helping supporting an equipment on the wearer's head, and in particular to a helmet-like head-mounting device that securely supports a vision correction/training device in front of the wearer's eyes with enhanced wearing comfort.

2. The Related Art

The special sense organs of human beings are located on the head, such as ears, eyes and nose. The capability of the sense organ deteriorates with age or be damaged by accidents. Examples of the deterioration of the sense organ are myopia and presbyopia. Myopia and presbyopia are often corrected by wearing glasses. Although up to now, cure of myopia is still not known, devices for reducing the progression or even restoring, at least partially, visual capability, are available in the market.

The eye training devices that are available in the market for training or correcting the eyesight of a user often comprises an optic device positionable in front of the user's eyes for causing movements of the eyeballs and the associated muscles to realize training of eyeballs and correction of eyesight. Such known devices often have a complicated structure and bulky size, making it difficult for its regular use, thus is inconvenient to the normal daily life.

Portable devices for training eyeballs are also available. Such portable devices allow a user to wear on the head to support optic elements in front of his or her eyes. However, a major drawback usually found in the conventional portable eye training devices is that the devices cannot be securely fixed on the wearer's head, leading to undesired movement of the device with respect to the head.

On the other hand, with the development of electronics, a lot of portable multi-media devices are available currently. Some of these devices provide head-mounted earphones for direct transmission of music or other sounds to the user's hears. This also requires a head-mounting device to help securing the earphones to the wearer's head. Thus, head-mounting devices that help mounting a device on the head of a wearer is commonly required nowadays.

Thus, the present invention is aimed to provide a head-mounting device that securely retains a device on a wearer's head so as to overcome the problems of the prior arts.

### SUMMARY OF THE INVENTION

Therefore, a primary objective of the present invention is to provide a head-mounting device that is securely mountable to a wearer's head without undesired relative movement with respect to the head.

Another objective of the present invention is to provide a head-mounting device that can be comfortably mounted to a wearer's head for a long while by providing enhanced air ventilation to reduce discomfort caused by wearing the device.

A further objective of the present invention is to provide a head-mounting device that securely retains an eye training device in front of a wearer's eyes.

To achieve the above objectives, in accordance with the present invention, there is provided a head-mounting device comprising an elongate strip-like body having an arcuate configuration suitable to fit over a wearer's head and extending from the forehead to the back of the head whereby front and rear ends of the strip-like body are located adjacent to the forehead and the back of the head. Two side-wings extend from opposite edges of the strip-like body in substantially opposite directions for engaging sides of the wearer's head. An electrically powered eye training device is attached to the front end of the strip-like body whereby the eye training device is substantially located in front of the wearer's eyes. A controller is attached to the rear end of the strip-like body and is electrically connected to the electrically powered eye training device for controlling the operation of the eye training device.

### BRIEF DESCRIPTION OF THE DRAWINGS

The present invention will be apparent to those skilled in the art by reading the following description of preferred embodiments thereof, with reference to the attached drawings, in which:

FIG. 1 is a perspective view of a head-mounting device constructed in accordance with the present invention with an eye training device attached thereto;

FIG. 2 is a front view of the head-mounting device shown in FIG. 1, together with the eye training device;

FIG. 3 is a rear view of the head-mounting device shown in FIG. 1, together with the eye training device;

FIG. 4 is a side elevational view of the head-mounting device shown in FIG. 1, together with the eye training device;

FIG. 5 is a front view showing the head-mounting device worn on a wearer's head; and

FIG. 6 is similar to FIG. 2 but showing a head-mounting device in accordance with another embodiment of the present invention.

### DETAILED DESCRIPTION OF THE PREFERRED EMBODIMENTS

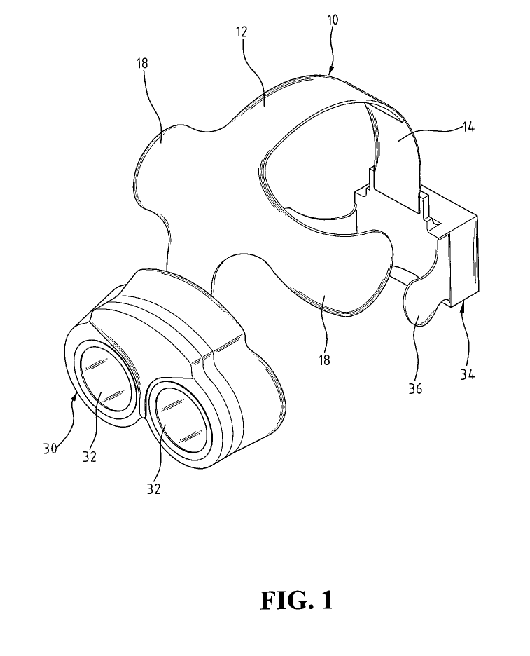

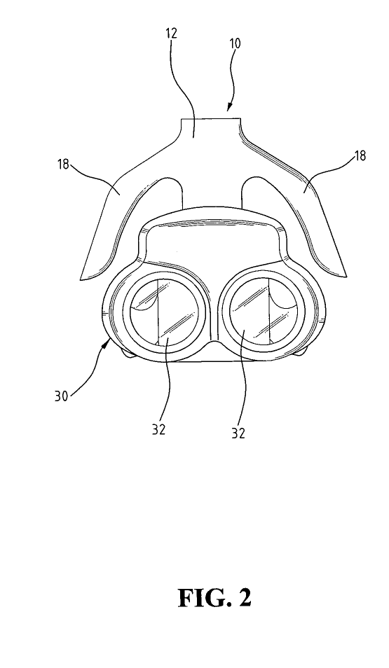

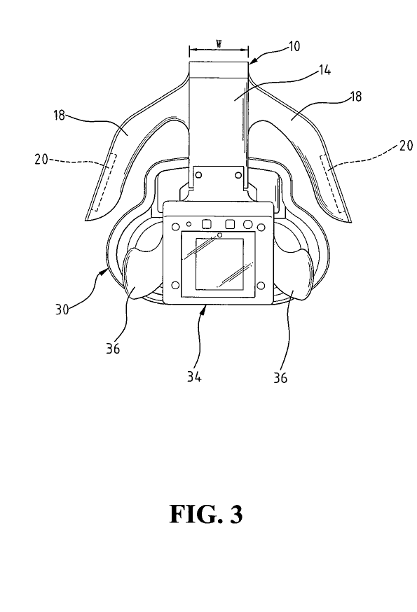

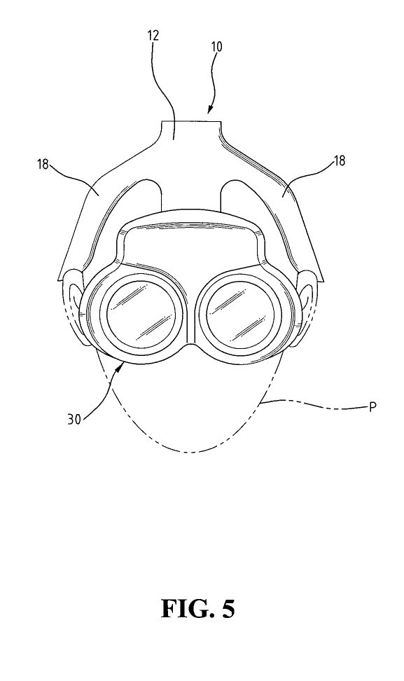

With reference to the drawings and in particular to FIGS. 1-4, a head-mounting device constructed in accordance with the present invention, generally designated with reference numeral 10, comprises an elongate strip-like body 12 that has a semi-circular or arcuate configuration substantially corresponding to the contour of a wear's head P (see FIG. 5) whereby the strip-like body 12 can be fit over and thus mounted to the top of the wearer's head P. The strip-like body 12 has opposite ends substantially located adjacent to the forehead and the back of the head with the strip-like body 12 extending therebetween across the top of the wearer's head P.

The strip-like body 12 is comprised of at least a flexible or deformable section for compliantly fitting over the wearer's head P and making the wearer comfortable. In the embodiment illustrated, the strip-like body 12 comprises a front section (not labeled) and a rear section 14 that is made of a flexible material, such as soft metal and elastic material, and is thus deformable for adjustment to compliantly fit over the wearer's head P. Apparently, if desired, the strip-like body 12 can be comprised of alternating rigid sections and flexible/deformable sections to securely and comfortably mount to the wearer's head P.

The strip-like body 12 may assume any width w as long as it does not cause discomfort to the wearer, such as that caused by force concentrated on a small thin area.

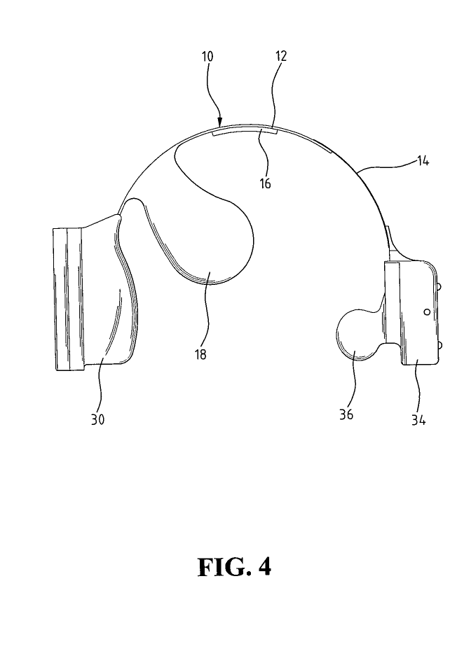

A cushion pad 16 made of resilient material or soft material is attached to an inside surface (not labeled) of the strip-like body 12 that contacts the wearer's head P. Thus, a comfortable engagement may be provided between the strip-like body 12 and the wearer's head P. The cushion pad 16 may occupy only a portion of the inside surface of the strip-like body 12 as illustrated in FIG. 4. Alternatively, the cushion pad 16 may be coextensive with the inside surface of the strip-like body 12. The width of the cushion pad 16 can be the same as or smaller than that of the strip-like body 12

If desired, there may be two or more cushion pads 16 spaced along the inside surface of the strip-like body 12.

Two side-wings 18 extend from opposite edges of the strip-like body 12. Preferably, the side-wings 18 are integrally formed with the strip-like body 12. The side-wings 18 are of arcuate configuration and engage with sides of the wearer's head P to securely retain the head-mounting device 10 on the head P in a comfortable manner.

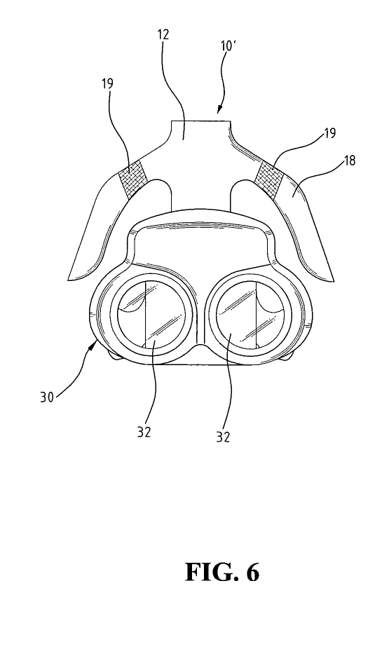

If desired, the side-wings 18 can be made separate from the strip-like body 12 and connected to the strip-like body 12 by deformable or flexible sections 19 (see FIG. 6). The provision of the deformable or flexible sections 19 allows for change of angular position of the side-wings 18 with respect to the strip-like body 12 for more securely retaining the head-mounting device 10 on the wearer's head P. In addition, the deformability of flexibility of the sections 19 provides resiliency for securely attaching the side-wings 18 to the sides of the wearer's head P. Thus, by positioning the strip-like body 12 over the top of the wearer's head P and having the side-wings 18 firmly engaging the sides of the wearer's head P, the head-mounting device 18 is securely mounted on the wearer's head P. No sliding or accidental drop-off of the head-mounting device 10 from the wearer's head P may occur.

Additional cushion pads 20 are provided on inside surfaces of the side-wings 18 for comfortable contact between the side-wings 18 and the wearer's head P.

Since the head-mounting device 10 of the present invention does not completely cover and thus shield the top portion of the wearer's head P, which is commonly observed in a motorcyclist's helmet, enhanced air ventilation can be realized for long term and comfortable wearing of the device 10.

In the embodiment illustrated, the head-mounting device 10 carries a front-end device attached to a front end of the strip-like body 12. In the embodiment illustrated, the front-end device comprises an eye training device, which is designated with reference numeral 30, whereby when the head-mounting device 10 is worn on the wearer's head P, the eye training device 30 is substantially located in front of eyes of the wearer as illustrated in FIG. 5. An example of the eye training device 30 comprises an electrically powered device comprising two light-transmitting windows 32 in which optic elements are arranged for guiding and controlling light projecting onto the eyes. The optic elements are controlled and driven by electrical motors or other electrically powered driving devices in order to realize the control of the light projecting onto the eyes for performing eye training. Taiwan Patent Application No. 91116074 filed on Jul. 19, 2002 discloses an example of the eye training device 30.

A rear-end device is selectively attached to a rear end of the strip-like body 12 for force balance with respect to the front-end device 30. To be positioned substantially in front of the wearer's eyes, the eye training device 30 that is the front-end device in the embodiment illustrated, is attached to the front end of the strip-like body 12 that is located at the forehead of the wearer. The weight of the eye training device 30 may cause the head-mounting device 10 to slide frontward. The rear-end device is thus comprised of a count weight 34 selected to balance the weight of the eye training device 30. The count weight 34 is selected to balance the weight of the eye training device 30 so that no undesired sliding or movement of the head-mounting device 10 may occur.

In addition, in the example illustrated, the rear-end device is comprised of a casing inside which electrical driving and control circuit and associated elements, as well as a power source, such as a battery set, if desired, are fixed. The circuit is connected to the electrically powered eye training device 30 via for example wires or similar conductors extending along the strip-like body 12. Thus, the rear-end device, not only providing a count weight 34, also functions as a controller of the eye training device 30.

Rear-wings 36 are formed on opposite sides of the rear-end device 34 and extend circumferentially along and engage the wearer's head P. The rear-wings 36 help to more firmly secure the head-mounting device 10 on the wearer's head P. Although only rear-wings 36 extending from the rear-end device 34 are shown, it is obvious to those having ordinary skills to add front wings (not shown) extending from the front-end device, namely the eye training device 30, for further firmly securing the head-mounting device 10 on the wearer's head P.

Preferably, the rear-wings 36 are integrally formed with the rear-end device 34. However, the rear-wings 36 may be separately made and connected to the rear-end device by means of deformable members (not shown) for compliantly engaging the sides of the wearer's head P.

In the embodiment illustrated, since there is no front wings, the side-wings 18 are arranged close to the front-end device 30 in order to be substantially opposite to the rear-wings 36, as best seen in FIG. 4. Apparently, the size, position and configuration of the side-wings 18 can be adjusted in accordance with practical requirements for different applications.

Referring to FIG. 6, a head-mounting device in accordance with another embodiment of the present invention is shown, generally designated with reference numeral 10′. The head-mounting device 10′ is substantially the same as the head-mounting device 10 discussed with reference to FIGS. 1-4, except that the side-wings 18 of the head-mounting device 10′ are separately made and are connected to the opposite edges of the strip-like body 12 by deformable or flexible sections 19, which allows for adjustment of the angular position of the side-wings 18 with respect to the strip-like body 12 for more firmly attaching the head-mounting device 10′ to the wearer's head P.

To this point, advantages of the head-mounting device 10, 10′ in accordance with the present invention are summarized as follows:
(1) The head-mounting device 10, 10′ can be securely mounted to the wearer's head P.
(2) The head-mounting device 10, 10′ does not completely cover and shield the wearer's head P and thus has enhanced air ventilation. Thus, long term continuous wearing does not cause any discomfort to the wearer and it is particular suitable for applications that requires long term wearing of the device, such as eye training applications.
(3) The head-mounting device 10, 10′ has a simple structure and thus low costs.
(4) Due to the simple structure, the head-mounting device 10, 10′ is light weighted.
(5) The head-mounting device 10, 10′ is particularly suitable for carrying an eye training device for long term continuous therapy of eye troubles.

Although the present invention has been described with reference to the preferred embodiments thereof, it is apparent to those skilled in the art that a variety of modifications and changes may be made without departing from the scope of the present invention which is intended to be defined by the appended claims.

1. A head-mounting device comprising:
an elongate strip-like body having an arcuate configuration adapted to be fit over a wearer's head and extending from forehead to back of the head; and
two side-wings extending from opposite edges of the strip-like body in substantially opposite directions for engaging sides of the wearer's head;
wherein the strip-like body comprises a flexible deformable section for compliantly fitting over and tightly engaging the wearer's head.

2. The head-mounting device as claimed in claim 1, wherein the strip-like body is fit over the head so that front and rear ends of the strip-like body are respectively located at the forehead and back of the head and wherein the head-mounting device further comprises a front-end device attached to the front end of the strip-like body, the front-end device comprising an eye training device substantially located in front of eyes of the wearer.

3. The head-mounting device as claimed in claim 2 further comprising a rear-end device mounted to a rear end of the strip-like body for weight balancing with respect to the front-end device.

4. The head-mounting device as claimed in claim 3, wherein the front-end device comprises an electrically powered device and wherein the rear-end device comprises a casing inside which an electrical controller is arranged and electrically connected to the electrically powered device of the front-end device.

5. The head-mounting device as claimed in claim 3 further comprising rear-wings extending from opposite sides of the read end device and extending circumferentially along the head.

6. The head-mounting device as claimed in claim 1 further comprising rear-wings extending from opposite sides of a read end of the strip-like body that is located at the back of the head and extending circumferentially along the head.

7. The head-mounting device as claimed in claim 1 further comprising a cushion pad attached to an inside surface of the strip-like body to provide a comfortable engagement between the strip-like body and the head.

8. The head-mounting device as claimed in claim 1 further comprising a cushion pad attached to each side-wing to provide a comfortable engagement between the side-wing and the head.

9. The head-mounting device as claimed in claim 1 further comprising a deformable flexible section connecting each side-wing to the corresponding edge of the strip-like body.

10. A head-mounting device comprising:
an elongate strip-like body having an arcuate configuration adapted to be fit over a wearer's head, the strip-like body having a predetermined width and front and rear ends respectively located adjacent to forehead and back of the head when the strip-like body is fit over the head;
two side-wings extending from opposite edges of the strip-like body in substantially opposite directions for engaging sides of the wearer's head;
an electrically powered eye training device attached to the front end of the strip-like body to be substantially located in front of eyes of the wearer; and
an electrical controller attached to the rear end of the strip-like body and electrically connected to the electrically powered eye training device.

11. The head-mounting device as claimed in claim 10, wherein the strip-like body comprises at least one deformable flexible section for compliantly fitting over and thus tightly engaging the wearer's head.

12. The head-mounting device as claimed in claim 10 further comprising rear-wings extending from opposite sides of the electrical controller and extending circumferentially along the head.

13. The head-mounting device as claimed in claim 10 further comprising a cushion pad attached to an inside surface of the strip-like body to provide a comfortable engagement between the strip-like body and the head.

14. The head-mounting device as claimed in claim 10 further comprising a cushion pad attached to each side-wing to provide a comfortable engagement between the side-wing and the head.

15. The head-mounting device as claimed in claim 10 further comprising a deformable flexible section connecting each side-wing to the corresponding edge of the strip-like body.
